SEQUENCE LISTING (31 sequences)

SEQ ID NO: 1 - DNA, 894 bp, Artificial Sequence
  BpGH16A coding sequence
gcagaaaatttaaataataaatcatacgagtgggacatctatcctgtaccagctaatgctggtgacggtatggtgtggaaacttcatccacagtcggacgactttaattatattgctgacgaaaaggataaaggaaaagagttctatgccaaatggactgatttctatcataatcattggacagggcctgctcctacaatatggcagagagaccatgtttccgtttcagacggattccttaaaatcagagccagccgtcctgaagatgtccctctaaagaaagttgtaagcggacctaacacaaaggaactcccgggaacctatacaggatgtattacatcgaagactcgtgtaaaatatccggtttatgtagaagcatacgccaaactttcaaattcaaccatggcatccgatgtatggatgcttagccctgatgatactcaggaaatcgatattatagaggcatacggtggtgatagagacggtggaggttatggtgccgacagacttcacttaagccatcacatattcatccgtcagccattcaaggattatcagccaaaggattcaggttcatggtataaggatgacaagggaacattgtggcgcgatgattttcatcgtgttggagtattctggaaagatcctttcacacttgaatattatgtagatggagaactcgtcagaactataagcggtaaggatattattgatcccaacaactacactggtggcacggggttggttaaggatatggacatcataataaatatggaagaccaaagctggagggccgttaaaggtttaagccctacggatgaggaactgaaaaatgtggaagatcacactttccttgtcgactggataagggtttatacactggtcccagaagaa

SEQ ID NO: 2 - DNA, 54 bp, Artificial Sequence
  CL signal paptide  coding sequence
atgaggtctttgctaatcttggtgctttgcttcctgcccctggctgctctgggg

SEQ ID NO: 3 - DNA, 267 bp, Artificial Sequence
  alpha-MF signal paptide  coding sequence
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 4 - DNA, 1074 bp, Artificial Sequence
  STA1 signal paptide  coding sequence
atggtaggcctcaaaaatccatatacgcacactatgcaaagaccatttctactcgcttatttggtcctttcgcttctatttaactcagctttgggttttccaactgcactagttcctagaggatcctcctctagcaacatcacttcctccggtccatcttcaactccattcagctctgctactgaaagcttttctactggcactactgtcactccatcatcatccaaataccctggcagtaaaacagaaacttctgtttcttctacaaccgaaactaccattgttccaactacaactacgacttctgtcataacaccatcaacaaccactattaccactacggtttgctctacaggaacaaactctgccggtgaaactacttctggatgctctccaaagaccattacaactactgttccatgttcaaccagtccaagcgaaaccgcatcggaatcaacaaccacttcacctaccacacctgtaactacagttgtctcaaccaccgtcgttactactgagtattctactagtacaaaacaaggtggtgaaattacaactacatttgtcaccaaaaacattccaaccacttacctaactacaattgctccaacttcatcagtcactacggttaccaatttcaccccaaccactattactactacggtttgctctacaggaacaaactctgccggtgaaactacctctggatgctctccaaagactgtcacaacaactgttccttgttcaactggtactggcgaatacactactgaagctaccgcccctgttacaacagctgtcacaaccaccgttgttaccactgaatcctctacgggtactaactccgctggtaagacgacaactagttacacaacaaagtctgtaccaaccacctatgtatttgactttggcaagggcattctcgatcaaagctgcggcggtgtattttcaaacaacggctcttcgcaagtgcagctgcgggatgtagtcttgatgaatgggacagtggtatacgattcaaacggcgcttgggacagtagtgcgctggaggagtggctccagcgacagaaaaaa

SEQ ID NO: 5 - DNA, 57 bp, Artificial Sequence
  SED1 signal paptide coding sequence
atgaaattatcaactgtcctattatctgccggtttagcctcgactactttggcccaa

SEQ ID NO: 6 - DNA, 20 bp, Artificial Sequence
  gHIS3_F
tccacctagcggatgactct

SEQ ID NO: 7 - DNA, 20 bp, Artificial Sequence
  gHIS3_R
tgcattaccttgtcatcttc

SEQ ID NO: 8 - DNA, 60 bp, Artificial Sequence
  dDNA_HIS3_F
gtaaagcgtattacaaatgaaaccaagattcagattgcgatctctttaaagggttaaccc

SEQ ID NO: 9 - DNA, 60 bp, Artificial Sequence
  dDNA_HIS3_R
ttctgggaagatcgagtgctctatcgctaggggttaaccctttaaagagatcgcaatctg

SEQ ID NO: 10 - DNA, 41 bp, Artificial Sequence
  16A_W/OSP_F_SpeI
ataactagtgcagaaaatttaaataataaatcatacgagtg

SEQ ID NO: 11 - DNA, 33 bp, Artificial Sequence
  16A_W/OSP_R_XhoI
atactcgagttcttctgggaccagtgtataaac

SEQ ID NO: 12 - DNA, 90 bp, Artificial Sequence
  16A_CL_F_SpeI
ataactagtatgaggtctttgctaatcttggtgctttgcttcctgcccctggctgctctgggggcagaaaatttaaataataaatcatac

SEQ ID NO: 13 - DNA, 25 bp, Artificial Sequence
  16A_CL_R_XhoI
atactcgagttcttctgggaccagt

SEQ ID NO: 14 - DNA, 34 bp, Artificial Sequence
  alpha-MF_F_SpeI
ataactagtatgagatttccttcaatttttactg

SEQ ID NO: 15 - DNA, 40 bp, Artificial Sequence
  alpha-MF_R_16A
gcagaaaatttaaataataaagcttcagcctctcttttct

SEQ ID NO: 16 - DNA, 49 bp, Artificial Sequence
  16A_F_ alpha-MF
gagaaaagagaggctgaagctgcagaaaatttaaataataaatcatacg

SEQ ID NO: 17 - DNA, 29 bp, Artificial Sequence
  16A_R_alpha-MF_BamHI
ataggatccttcttctgggaccagtgtat

SEQ ID NO: 18 - DNA, 28 bp, Artificial Sequence
  STA1_F_EcoRI
atagaattcatggtaggcctcaaaaatc

SEQ ID NO: 19 - DNA, 49 bp, Artificial Sequence
  STA1_16A_ R
gcagaaaatttaaataataaatcatacgagttttttctgtcgctggagc

SEQ ID NO: 20 - DNA, 50 bp, Artificial Sequence
  16A_STA1_ F
ggctccagcgacagaaaaaagcagaaaatttaaataataaatcatacgag

SEQ ID NO: 21 - DNA, 29 bp, Artificial Sequence
  16A_ R_XhoI
atactcgagttcttctgggaccagtgtat

SEQ ID NO: 22 - DNA, 89 bp, Artificial Sequence
  16A_SED1_F_SpeI
ataactagtatgaaattatcaactgtcctattatctgccggtttagcctcgactactttggcccaagcagaaaatttaaataataaatc

SEQ ID NO: 23 - DNA, 24 bp, Artificial Sequence
  16A_SED1_R_XhoI
atactcgagttcttctgggaccag

SEQ ID NO: 24 - DNA, 71 bp, Artificial Sequence
  dDNA-CS5-F
aaaagagaagaaaaaagagaagaaatgaattctattatgatagcgaatgcaattaaccctcactaaaggga

SEQ ID NO: 25 - DNA, 72 bp, Artificial Sequence
  dDNA-CS5-R
tgctggttgccttattaatttatatggaagacgagataattcattaattagtaatacgactcactatagggc

SEQ ID NO: 26 - DNA, 90 bp, Artificial Sequence
  dDNA-CS5+60_F
atggtacacgctcttggcaacattgaaattacagctctcatatataaaaaatggaaagaaaaaagagaagaaaaaagagaagaaatgaat

SEQ ID NO: 27 - DNA, 90 bp, Artificial Sequence
  dDNA-CS5+60_R
ggcataacaatagcgcacagatccgcaggtttcgtaatacgcttaacaataggcgtctcctgctggttgccttattaatttatatggaag

SEQ ID NO: 28 - DNA, 43 bp, Artificial Sequence
  gCS5_F
ctggtagttgcacagaaagagttttagagctagaaatagcaag

SEQ ID NO: 29 - DNA, 42 bp, Artificial Sequence
  gCS5_R
tctttctgtgcaactaccagcgatcatttatctttcactgcg

SEQ ID NO: 30 - DNA, 27 bp, Artificial Sequence
  Conf-CS5-F
aatgaattctattatgatagcgaatgc

SEQ ID NO: 31 - DNA, 19 bp, Artificial Sequence
  Conf-CS5-R
cacaggatttacgaagacc

(19) United States
(12) Patent Application Publication
(10) Pub. No.: US 2024/0294891 A1
(43) Pub. Date: Sep. 5, 2024

(54) RECOMBINANT YEAST SACCHAROMYCES BOULARDII FOR PRODUCTION OF NEOAGAROOLIGOSACCHARIDE AND USE THEREOF

(71) Applicant: KOREA UNIVERSITY RESEARCH AND BUSINESS FOUNDATION, Seoul (KR)

(72) Inventors: Kyoung Heon KIM, Seoul (KR); So Ra YU, Seoul (KR); Ye Rin JIN, Seoul (KR); Eun Ju YUN, Seoul (KR)

(73) Assignee: KOREA UNIVERSITY RESEARCH AND BUSINESS FOUNDATION, Seoul (KR)

(21) Appl. No.: 18/571,437

(22) PCT Filed: Jun. 20, 2022
(86) PCT No.: PCT/KR2022/008703

§ 371 Date: Feb. 27, 2024

(30) Foreign Application Priority Data
Jun. 18, 2021 (KR) 10-2021-0079055

(51) Int. Cl. C12N 9/38; C12N 15/81; C12P 19/02; C12P 19/14

(52) U.S. Cl. CPC C12N 9/2468; C12N 15/81; C12P 19/02; C12P 19/14; C12Y 302/01081

(57) ABSTRACT

The present invention relates to a strain that produces neoagarooligosaccharides from agarose, which is a representative polysaccharide constituting red algae, using Saccharomyces boulardii, which is a probiotic yeast, a production method thereof, and a use thereof. The present invention aims to achieve synbiotics through the production of prebiotic substances using probiotic strains and can be used as an intestinal microbial factory.

### CROSS-REFERENCE TO RELATED APPLICATION

This application claims priority to and the benefit of Korean Patent Application No. 10-2021-0079055, filed on Jun. 18, 2021, the disclosure of which is incorporated herein by reference in its entirety.

### BACKGROUND

### 1. Field of the Invention

The present invention relates to a recombinant strain that produces neoagarooligosaccharids from agarose, which is a representative polysaccharide constituting red algae, and a use thereof.

### 2. Discussion of Related Art

Saccharomyces boulardii is a generally recognized as safe (GRAS) non-pathogenic yeast first discovered in the peels of lychee and mangosteen. It is known that Saccharomyces boulardii can survive in the human gastrointestinal tract due to its high tolerance to low pH and heat (Douradinha B., et al. (2014) Bioengineered. 5, 21-29, Czerucka D., et al. al. (2007) Aliment. Pharmacol. Ther. 26(6), 767-78). Saccharomyces boulardii is the only probiotic yeast found to be effective in double-blind studies.

Unlike digestible food, non-digestible food reaches the large intestine and is utilized by the intestinal microbial community. As a result, non-digestible food changes the intestinal environment and affects overall human health (Pistollato F., et al. (2016) Nutr Rev. 74, 624-634, Sonnenburg E. D., et al. (2014) Cell Metab. 20, 779-786). Agarose, which is a representative polysaccharide constituting red algae, is one of the non-digestible foods commonly found in East Asia (Kolb N., et al. (2004) Food Technol Biotechnol. 42, 57-61). Agarose is decomposed into neoagarooligosaccharides such as neoagarotetraose and neoagarobiose by endo-type beta-agarase, and the neoagarooligosaccharides have been reported to have various physiological and biological functions such as anti-obesity, anti-diabetic, anti-inflammatory, anti-tumor activity, and prebiotic effects (Torres M. D., et al. (2019) Mar Drugs. 17, 314, Hong S. J., et al. (2017) Mar Drugs. 15, 90, Lee M. H., et al. (2017) BMB Rep. 50, 263, Lin F., et al. (2019) Mar Drugs. 17, 154, Wang W., et al. (2017) Sci Rep. 7, 442-52, Kim M., et al. (2020) Biomaterials. 263, 120391, Hu G., et al. (2006) Anaerobe. 12, 260-266). In particular, the tetrasaccharide neoagarotetraose, which is a type of neoagarooligosaccharide, was also found to have various functional properties such as anti-inflammatory and antioxidant activity, and prebiotic effects (Zhang N., et al. (2017) Food Agric Immunol. 28, 1408-1423, Xu X-Q., et al. (2018) Food Chem. 240, 330-337).

At this time, when an intestinal microbial factory that can produce useful proteins such as prebiotics directly in the intestines is developed, more accurate research on the impact of intestinal microorganisms on health will become possible, and furthermore, it may be used to treat actual diseases.

However, there have been no reports of successful research in producing neoagarooligosaccharides that can be used as prebiotics from red algae agar or agarose in the yeast Saccharomyces boulardii, which is a eukaryotic cell and a probiotic.

### RELATED ART DOCUMENT

### Patent Document

Korean Patent Registration No. 10-1864800

### SUMMARY OF THE INVENTION

The present invention is directed to providing a probiotic recombinant yeast capable of producing prebiotic substances in the intestines.

The present invention is also directed to providing a composition for producing a prebiotic including a probiotic recombinant yeast and a substrate, and a production method thereof.

In order to produce prebiotic substances from probiotic yeast using metabolic engineering technology, the present inventors attempted to produce neoagarooligosaccharides including neoagarotetraose for the first time by introducing a Bacteroides plebeius-derived enzyme BpGH16A gene into the yeast Saccharomyces boulardii. The present inventors completed the present invention by quantifying neoagarooligosaccharides produced after fermenting Saccharomyces boulardii with agarose using a method of quantifying neoagarooligosaccharides utilizing high-performance liquid chromatography (HPLC) and preparing Saccharomyces boulardii, which is a probiotic yeast that can express BpGH16A, which is a beta-agarase, and secrete the enzyme outside the strain.

Accordingly, the present invention provides recombinant Saccharomyces boulardii transformed with a gene encoding beta-agarase.

The purpose of the recombinant Saccharomyces boulardii of the present invention is to ultimately use it to produce neoagarooligosaccharides using agarose as a substrate. However, since Saccharomyces boulardii cannot absorb agarose as a substrate into the strain and metabolize it, a means is required to secrete the beta-agarase enzyme, which can decompose agarose into neoagarooligosaccharides, outside the strain. Therefore, the recombinant Saccharomyces boulardii of the present invention may be transformed with a gene encoding a signal peptide capable of secreting the beta-agarase outside the strain.

The signal peptide plays a role in secreting beta-agarase, specifically an enzyme expressed from the BpGH16A gene represented by SEQ ID NO: 1 in Saccharomyces boulardii, and may be one or more of a chicken lysozyme signal peptide (CL), a Saccharomyces cerevisiae-derived α-binding factor signal peptide (α-MF), a Saccharomyces diastaticus-derived STA1 signal peptide (STA1), and a Saccharomyces cerevisiae-derived SED1 signal peptide (SED1). In one specific example, it was confirmed that when SED1 was used as the signal peptide, a secretion amount of enzyme and a production amount of neoagarooligosaccharides were the best.

The chicken lysozyme signal peptide (CL) may be represented by SEQ ID NO: 2, the Saccharomyces cerevisiae-derived α-binding factor signal peptide (α-MF) may be represented by SEQ ID NO: 3, the Saccharomyces diastaticus-derived STA1 signal peptide (STA1) may be represented by SEQ ID NO: 4, and the Saccharomyces cerevisiae-derived SED1 signal peptide (SED1) may be represented by SEQ ID NO: 5.

The transformation of Saccharomyces boulardii with a gene encoding the beta-agarase and/or signal peptide can be performed using a recombinant vector including the gene or CRISPR-Cas9, which is a genetic recombination technology.

When using a recombinant vector such as a plasmid for the transformation of the Saccharomyces boulardii, auxotrophic mutants, for example, a mutant Saccharomyces boulardii strain in which one or more of the HIS3, TRP1 and URA3 genes are inactivated may be used.

In addition, the present invention provides a composition for producing prebiotics including the recombinant Saccharomyces boulardii and agarose as a substrate.

The recombinant Saccharomyces boulardii of the present invention can produce prebiotics by secreting beta-agarase outside the strain and decomposing agarose as a substrate, and in addition, Saccharomyces boulardii is a probiotic strain and may provide prebiotics and probiotics at the same time.

Accordingly, the present invention provides a synbiotic composition including the recombinant Saccharomyces boulardii and agarose as a substrate.

The prebiotics may be neoagarooligosaccharides, and specifically, may be neoagarobiose, neoagarotetraose, and neoagarohexaose, which are produced through the decomposition of agarose.

In addition, the composition may further include other probiotics in addition to the Saccharomyces boulardii strain, specifically, may include strains belonging to the Lactobacillus genus, Bifidobacterium genus, and Enterococcus genus, and more specifically, may include any probiotic strain that can ingest and metabolize neoagarooligosaccharides without limitation.

In addition, the present invention includes a method of producing neoagarooligosaccharides, including: reacting the culture fluid or extract of the recombinant Saccharomyces boulardii with agarose as a substrate; and separating and purifying neoagarooligosaccharides from the product of the above step

The description of the recombinant Saccharomyces boulardii and neoagarooligosaccharide is omitted to prevent duplication with the above-mentioned content.

Specifically, the culture or fermentation of the strain to produce beta-agarase from the recombinant Saccharomyces boulardii of the present invention is performed at a temperature of 20 to 40° C., preferably 30 to 40° C. for 1 to 7 days, preferably for 2 to 5 days.

### BRIEF DESCRIPTION OF THE DRAWINGS

FIG. 1 shows an overall schematic diagram of neoagarotetraose production by Saccharomyces boulardii engineered using metabolic engineering technology. The introduction of a BpGH16A gene, which is a beta-agarase, was performed using two different systems: a vector system with an auxotrophic marker and a CRISPR-Cas9 system.

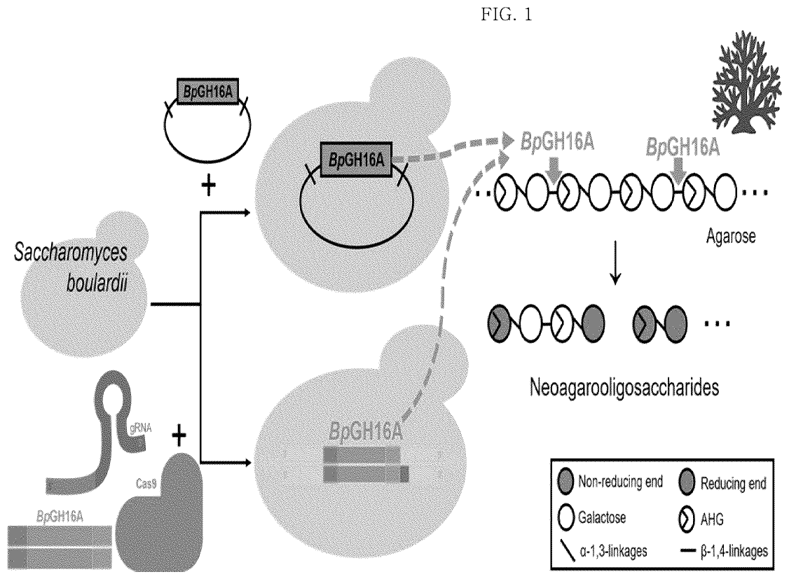

FIG. 2 shows the results of confirming the fermentation product using a strain (SB-HTU_16A_C) into which the BpGH16A gene was introduced using HPLC. Fermentation was performed at 37° C. for 72 hours using 2.5 g/L of agarose as a substrate. (A) is the result of fermentation product analysis using a wild-type strain as a control group, and (B) is the result of fermentation product analysis using a strain (SB-HTU_16A_C) into which the BpGH16A gene and a chicken lysozyme signal peptide sequence were introduced. Neoagarotetraose (NeoDP4) was detected as a peak at a retention time of approximately 7.6 minutes.

FIG. 3 is the result of comparing the amounts of neoagarotetraose (NeoDP4) produced when strains, in which different signal peptide sequences were introduced in front of the BpGH16A gene, which is a beta-agarase, in Saccharomyces boulardii were fermented with agarose. Fermentation was performed at 37° C. for 72 hours using 2.5 g/L of agarose as a substrate. (A) is the result of analyzing the fermentation product using TLC, and (B) is the result of analyzing the fermentation product using HPLC and calculating the concentration.

FIG. 4 shows the results of confirming the materials and cell growth generated over time when fermentation was performed at 37° C. for 72 hours using agarose as a substrate using yeast (SB-HTU_16A_D) prepared based on a plasmid. (A) is the result of analyzing fermentation products for 72 hours using TLC. (B) shows the results of HPLC analysis and quantification of cell density and concentrations of glucose, ethanol, acetic acid, and neoagarotetraose during fermentation for 72 hours.

FIG. 5 relates to a method of preparing Saccharomyces boulardii, which can produce neoagarooligosaccharides, using CRISPR-Cas9 technology. (A) is an overall schematic diagram of the process of introducing beta-agarase into a strain using a CRISPR-Cas9 system. (B) is the result of introducing beta-agarase into the strain using the CRISPR-Cas9 system and then confirming through colony PCR whether it was actually introduced.

FIG. 6 shows the results of confirming the materials and cell growth produced over time when agarose was provided as a substrate using Saccharomyces boulardii (SB_16A_D) engineered using CRISPR-Cas9 technology and fermentation was performed for 72 hours. (A) is the result of analyzing fermentation products for 72 hours using TLC. (B) shows the results of HPLC analysis of cell density and concentrations of glucose, acetic acid, ethanol, and neoagarotetraose during fermentation for 72 hours.

### DETAILED DESCRIPTION OF EXEMPLARY EMBODIMENTS

Hereinafter, the present invention will be described in detail by way of Examples. The following Examples merely illustrate the present invention but do not limit the scope of the present invention.

### Example 1: Method of Preparing Plasmids and Strains

To use auxotrophic markers during strain production, strains with inactivated HIS3, TRP1, and URA3 genes were generated. The HIS3 gene was inactivated based on strain SB-TU in which TRP1 and URA3 were inactivated (Liu J-J., et al. (2016) Appl Environ Microbiol. 82, 2280-2287). The HIS3 gene was amplified using the primer pair gHIS3_F and gHIS3_R (Table 1), the resulting PCR product was digested by SacI and NotI, and ligated into the pRS42H vector to generate plasmid p42H_gHIS3 (Table 2). The repair DNA required after HIS3 inactivation was amplified by PCR using primers dDNA_HIS3_F and dDNA_HIS3_R (Table 1). Yeast transformation was performed using the PEG-LiAc method (Gietz R. D., et al. (1995) Yeast. 11, 335-360). After yeast transformation, Saccharomyces boulardii strain SB-HTU, in which HIS3, TRP1, and URA3 genes were inactivated, was prepared and used in the experiment (Table 3).

Plasmid construction to select an optimal signal peptide for beta-agarase secretion was performed as follows. The gene BACPLE_01670 encoding BpGH16A was cloned into the pRS426GPD plasmid. The BpGH16A gene fragment was amplified by PCR from genomic DNA of Bacteroides plebeius DSM 17135 (DSMZ, Braunschweig, Germany) using different primer pairs depending on the type of signal peptide (Table 1). A signal peptide sequence predicted at the N-terminus of BpGH16A was removed and then used. A total of four signal peptides, including a chicken lysozyme signal peptide (CL), a Saccharomyces cerevisiae-derived α-binding factor signal peptide (α-MF), a Saccharomyces diastaticus-derived STA1 signal peptide (STA1), and a Saccharomyces cerevisiae-derived SED1 signal peptide (SED1), were compared (Liu J-J., et al. (2016) Appl Environ Microbiol. 82, 2280-2287, Inokuma K., et al. (2016) Biotechnol Bioeng. 113, 2358-2366, Yanagisawa M., et al. (2016) Enzyme Microb Technol. 85, 82-89). In addition, to construct a control strain without a signal peptide, PCR was performed using the primer pair 16A_W/OSP_F_Spel and 16A_W/OSP_R_Xhol (Table 1). Plasmids such as p426_Bp_W/OSP, p426_Bp_CL, p426_Bp_aMF, p426_Bp_STA1, and p426_Bp_SED1 were generated, and yeast transformation using SB-HTU was performed using the PEG-LiAc method. That is, strains SB-HTU_16A_C, SB-HTU_16A_A, SB-HTU_16A_S, and SB-HTU_16A_D were prepared for a signal peptide comparison experiment (Table 3). As a control strain, SB-HTU_E, which includes only the pRS426GPD vector without BpGH16A and a signal peptide, and SB-HTU_W, which includes the BpGH16A gene but no signal peptide, were prepared and used.

For stable expression of BpGH16A, guide RNA plasmid p42K_CS5 was used to integrate the BpGH16A gene into the genome of Saccharomyces boulardii (Table 2). p42K_CS5 was generated by inverse PCR of the pRS42K plasmid including a guide RNA sequence using the primer pair gRNA_CS5_F and gRNA_CS5_R (Table 1). The 20-bp targeting sequence of the guide RNA binds to the front of the PAM sequence (NGG) at an empty locus on chromosome XV (CS5). BpGH16A and SED1 signal peptides were integrated by homologous recombination without affecting the function of other genes. For homologous recombination, plasmid p426_16A_D was amplified using the primer pair dDNA-CS5-F and dDNA-CS5-R as donor DNA for CRISPR-Cas9-based genomic integration (Table 1). To overcome inefficiencies associated with genomic integration, PCR products constructed using the primer pair dDNA-CS5-F and dDNA-CS5-R were amplified once again by PCR using the primer pair CS5+60_F and CS5+60_R to increase a homology region to 120-bp (Table 1). In a yeast transformation process, 1 μg of Cas9-NAT, 20 μg of 16A-D-CS5, and 2 μg of p42K_CS5 were added to Saccharomyces boulardii and transformed using the PEG-LiAc method. Verification of genomic integration was performed by yeast colony PCR using the primer pair Conf-CS5-F and Conf-CS5-R (Table 1).

TABLE 1
List of primers used in the invention

| | Sequence (5′ →3′, restriction |
|---|---|
| Primer | sites are underlined) |
| gHIS3_F | TCCACCTAGCGGATGACTCT |
| gHIS3_R | TGCATTACCTTGTCATCTTC |
| dDNA_HIS3_F | GTAAAGCGTATTACAAATGAAACCAAGATTCAGATTGCG |
| | ATCTCTTTAAAGGGTTAACCC |
| dDNA_HIS3_R | TTCTGGGAAGATCGAGTGCTCTATCGCTAGGGGTTAACC |
| | CTTTAAAGAGATCGCAATCTG |
| 16A_W/OSP_ | ATA |
| F_SpeI | ACTAGTGCAGAAAATTTAAATAATAAATCATACGAGTG |
| 16A_W/OSP_ | ATA CTCGAGTTCTTCTGGGACCAGTGTATAAAC |
| R_XhoI | |
| 16A_CL_F_ | ATA |
| SpeI | ACTAGTATGAGGTCTTTGCTAATCTTGGTGCTTTGCTTCC |
| | TGCCCCTGGCTGCTCTGGGGGCAGAAAATTTAAATAATA |
| | AATCATAC |
| 16A_CL_R_XhoI | ATA CTCGAGTTCTTCTGGGACCAGT |
| αMF_F_SpeI | ATA ACTAGTATGAGATTTCCTTCAATTTTTACTG |
| αMF_R_16A | GCA GAA AAT TTA AAT AAT AAA GCT TCA GCC TCT CTT |
| | TTC T |
| 16A_F_aMF | GAG AAA AGA GAG GCT GAA GCT GCA GAA AAT TTA |
| | AAT AAT AAA TCA TAC G |
| 16A_R_αMF_ | ATA GGATCCTTCTTCTGGGACCAGTGTAT |
| BamHI | |
| STA1_F_EcoRI | ATA GAATTCATGGTAGGCCTCAAAAATC |
| STA1_16A_R | GCA GAA AAT TTA AAT AAT AAA TCA TAC GAG TTT TTT |
| | CTG TCG CTG GAG C |
| 16A_STA1_F | GGC TCC AGC GAC AGA AAA AAG CAG AAA ATT TAA |
| | ATA ATA AAT CAT ACG AG |
| 16A_R_XhoI | ATA CTCGAGTTCTTCTGGGACCAGTGTAT |
| 16A_SED1_F_ | ATA |
| SpeI | ACTAGTATGAAATTATCAACTGTCCTATTATCTGCCGGTT |
| | TAGCCTCGACTACTTTGGCCCAAGCAGAAAATTTAAATA |
| | ATAAATCA |
| 16A_SED1_R_ | ATA CTCGAGTTCTTCTGGGACCAG |
| XhoI | |
| dDNA-CS5-F | AAA AGA GAA GAA AAA AGA GAA GAA ATG AAT TCT |
| | ATT ATG ATA GCG AAT GCA ATT AAC CCT CAC TAA AGG |
| | GA |
| dDNA-CS5-R | TGC TGG TTG CCT TAT TAA TTT ATA TGG AAG ACG AGA |
| | TAA TTC ATT AAT TAG TAA TAC GAC TCA CTA TAG GGC |
| dDNA-CS5+60_F | ATG |
| | GTA CAC GCT CTT GGC AAC ATT GAA ATT ACA GCT |
| | CTC ATA TAT AAA AAA TGG AAA GAA AAA AGA GAA |
| | GAA AAA AGA GAA GAA ATG AAT |
| dDNA-CS5+60_R | GGC ATA ACA ATA GCG CAC AGA TCC GCA GGT TTC GTA |
| | ATA CGC TTA ACA ATA GGC GTC TCC TGC TGG TTG CCT |
| | TAT TAA TTT ATA TGG AAG |
| gCS5_F | CTG GTA GTT GCA CAG AAA GAG TTT TAG AGC TAG AAA |
| | TAG CAA G |
| gCS5_R | TCT TTC TGT GCA ACT ACC AGC GAT CAT TTA TCT TTC |
| | ACT GCG |
| Conf-CS5-F | AAT GAA TTC TAT TAT GAT AGC GAA TGC |
| Conf-CS5-R | CAC AGG ATT TAC GAA GAC C |

TABLE 2
List of plasmids used in the invention

| Plasmid | Description | Reference |
|---|---|---|
| pRS42H | 2μ origin | EUROSCARF |
| p42H_gHIS3 | pRS42H carrying HIS3 disruption gRNA | This study |
| | cassette | |
| pRS426GPD | URA3, GPD promoter, CYC1 terminator, 2μ | (Mumberg et al., |
| | origin, and Amp | 1995) |
| p426_Bp_W/OSP | pRS426GPD harboring BpGH16A from B. | This study |
| | plebeius, deletion signal peptide | |
| p426_Bp_CL | pRS426GPD harboring BpGH16A and chicken | |
| | lysozyme signal peptide | This study |
| p426_Bp_αMF | pRS426GPD harboring BpGH16A and α- | This study |
| | mating factor signal peptide | |
| p426_Bp_STA1 | pRS426GPD harboring BpGH16A and STA1 | This study |
| | signal peptide | |
| p426_Bp_SED1 | pRS426GPD harboring BpGH16A and SED1 | This study |
| | signal peptide | |
| Cas9-NAT | p414-TEF1p-Cas9-CYC1t-NAT1 | (Zhang et al., 2014) |
| pRS42K | 2μ origin, KanMX | (Taxis & Knop, |
| | | 2006) |
| p42K_CS5 | pRS42K, gRNA cassette targeting the | This study |
| | intergenic site on Chr XV | |
| 16A-D-CS5 | BpGH16A, SED1 signal peptide, donor DNA | This study |
| | for CS5 site integration | |

TABLE 3
List of strains used in the invention

| Strain | Description | Reference |
|---|---|---|
| S. boulardii | ATCC MYA-796 | ATCC |
| SB-TU | S. boulardii; TRP1 and URA3 disruption | (Liu et al., 2016) |
| SB-HTU | S. boulardii; HIS3, TRP 1, and URA3 disruption | This study |
| SB-HTU_16A_E | SB-HTU; pRS426GPD | This study |
| SB-HTU_16A_W | SB-HTU; BpGH16A, deletion signal peptide, | This study |
| | pRS426GPD | |
| SB-HTU_16A_C | SB-HTU; BpGH16A, chicken lysozyme signal | This study |
| | peptide, pRS426GPD | |
| SB-HTU_16A_A | SB-HTU; BpGH16A, a-mating factor signal | This study |
| | peptide, pRS426GPD | |
| SB-HTU_16A_S | SB-HTU; BpGH16A, STA1 signal peptide, | This study |
| | pRS426GPD | |
| SB-HTU_16A_D | SB-HTU; BpGH16A, SED1 signal peptide, | This study |
| | pRS426GPD | |
| SB_16A_D | S. boulardii; BpGH16A, SED1 signal peptide | This study |

### Example 2: Cell Culture Conditions

To produce neoagarooligosaccharides from the strain Saccharomyces boulardii prepared according to Example 1, 2.5 g/L of agarose was provided as a substrate at a temperature of 37° C. and fermentation was performed at 200 rpm in a 125-mL flask for 72 hours. To prevent coagulation of agarose during fermentation, agarose (Sigma-Aldrich) with a low gelation temperature was used. First, strains SB-HTU_16A_C, SB-HTU_16A_A, SB-HTU_16A_S, and SB-HTU_16A_D were cultured in yeast synthetic complete (YSC) medium at 37° C. and 200 rpm. Pre-cultured cells were centrifuged at 10,170×g for 10 minutes and washed twice with sterile distilled water, and harvested cells were inoculated into 20 mL of YSC medium containing 20 g/L of glucose and 2.5 g/L of agarose in 50 mM KHP buffer (pH 5.5). The initial cell density was inoculated at an optical density 600 nm (OD₆₀₀) of 1.0. As a control, fermentation of strains SB-HTU_16A_E and SB-HTU_16A_W was also performed under the same conditions.

### Example 3: Method of Quantifying Neoagarooligosaccharides Through Cell Growth Measurement and HPLC Analysis

Cell growth was measured by measuring OD₆₀₀ using a UV-visible spectrophotometer (Bio-Rad, Hercules, CA, USA). HPLC analysis was performed to analyze and quantify the reaction products of Saccharomyces boulardii and agarose, including neoagarotetraose, glucose, acetic acid, and ethanol. The column used during analysis was an Aminex HPX-87H column (Bio-Rad), which was equipped with a refractive index (RI) detector. The column and RI detector temperatures were set at 65° C. and 55° C., respectively, and the column used 0.005 M sulfuric acid as a mobile phase at a flow rate of 0.5 mL/min.

### Example 4: Method of Confirming Fermentation Products Through TLC Analysis

Thin layer chromatography (TLC) analysis was performed to identify the hydrolysis products of agarose during fermentation. For each time point (0, 12, 24, 36, 48, and 72-h) during fermentation, 1 mL of cell culture including fermentation products was obtained. For accurate measurements, the resulting cell culture was boiled to terminate further enzymatic reactions. After centrifugation at 16,609×g for 15 minutes at 4° C., 1 μL of each supernatant was loaded onto a silica gel 60 plate (Merck, Damstadt, Germany). After drying the TLC plate, it was visualized using a solution of 10% (v/v) sulfuric acid in ethanol and a solution of 0.2% (w/v) naphthoresorcinol in ethanol sequentially (Yun E. J., et al. (2013) Appl Microbiol Biotechnol. 97, 2961-2970).

### Example 5: Confirmation of Neoagarotetraose Production Using Saccharomyces boulardii

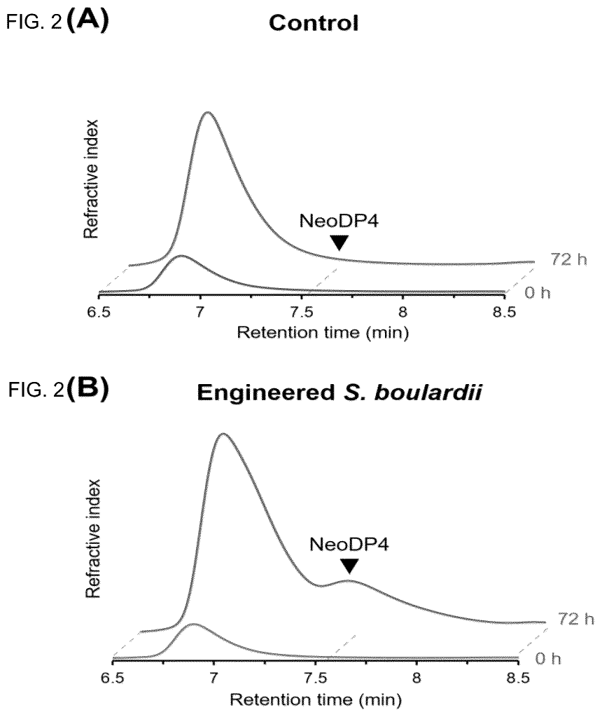

In order to produce neoagarotetraose using engineered yeast, secretion of beta-agarose, which enables agarose decomposition, is required in yeast. Therefore, the expression and secretion of endo-type beta-agarase BpGH16A by Saccharomyces boulardii were first tested. For testing, strain SB-HTU_16A_C was used, in which the chicken lysozyme signal peptide (CL), which was previously demonstrated to function in Saccharomyces boulardii (Liu J-J., et al. (2016) Appl Environ Microbiol. 82, 2280-2287), was introduced. In HPLC analysis of a SB-HTU_16A_C fermentation product, a peak was detected at a retention time of 7.6 minutes corresponding to neoagarotetraose (FIG. 2). In contrast, no peak was detected in the product of the control strain SB-HTU_16A_E including an empty vector. Therefore, these results confirmed that BpGH16A is functionally expressed, secreted from Saccharomyces boulardii, and produced neoagarotetraose by hydrolyzing agarose.

### Example 6: Selection of Optimal Signal Peptide to Produce Neoagarotetraose

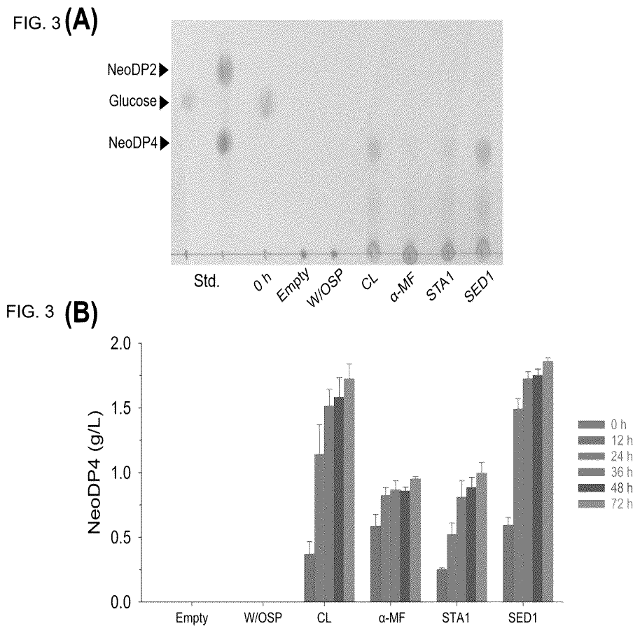

Since the enzyme BpGH16A was confirmed to be expressed and secreted in Saccharomyces boulardii, the next step was to find the optimal signal peptide to increase neoagarotetraose production. A total of four types of signal peptides: CL, α-MF, STA1, and SED1 were tested. Each signal peptide was fixed in front of the BpGH16A sequence and introduced into the SB-HTU strain to find the signal peptide that produces the most neoagarotetraose. The production of neoagarotetraose by the engineered yeast was confirmed by TLC analysis after 72 hours of culture (FIG. 3A). As a result, neoagarotetraose was noticeably produced by the strain with CL and SED1, slightly produced by the strain with STA1, and weakly detected in the strain with α-MF. To accurately compare the amount of neoagarotetraose generated by each engineered yeast, HPLC analysis was performed (FIG. 3B). As a result, neoagarotetraose was found to gradually increase as the 72-hour culture for each strain progressed, and the strain including SED1 produced the largest amount of neoagarotetraose. The amount of neoagarotetraose generated after 72 hours of fermentation was 1.73, 0.95, 0.99, and 1.86 g/L when signal peptides, namely CL, α-MF, STA1, and SED1, were used, respectively. Therefore, Saccharomyces cerevisiae-derived SED1, which produces the highest amount of neoagarotetraose, was selected as the optimal signal peptide for neoagarotetraose production.

Although neoagarotetraose was generated in all groups to which the signal peptide was attached, it was not generated in two control groups (FIG. 3). In particular, SB-HTU_16A_W, used as one of the negative controls, is a strain created to confirm that the extracellular activity of BpGH16A is derived from secretion by a heterologously expressed signal peptide rather than cell lysis. Since neoagarotetraose was not detected in the culture fluid of SB-HTU_16A_W, it was confirmed that agarose was decomposed to neoagarotetraose by BpGH16A secreted outside the cells. Since there has been no report on the comparison of signal peptides in Saccharomyces boulardii so far, there is a possibility that the present invention may be used in the field of protein production and secretion by Saccharomyces boulardii.

### Example 7: Fermentation to Produce Neoagarotetraose Using Improved Strains

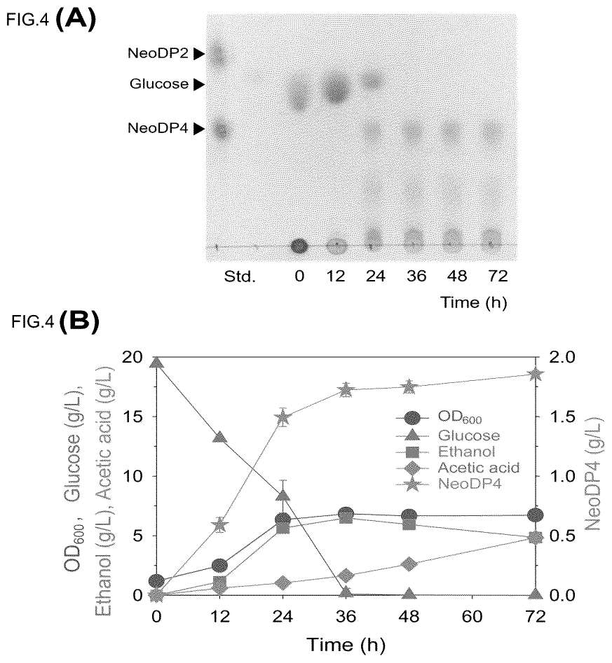

Based on the signal peptide selection results, strain SB-HTU_16A_D including the SED1 signal peptide was cultured. Fermentation was performed for 72 hours in YSC medium containing 2.5 g/L of agarose and without uracil. Neoagarotetraose production was confirmed by TLC analysis (FIG. 4A). It was confirmed that the initially added glucose was depleted after 36 hours. As a result of analyzing the fermentation products at each time point by HPLC, 1.86 g/L of neoagarotetraose was produced after 72 hours of fermentation (FIG. 4B). Cell growth entered a stationary phase at 24 hours and reached OD₆₀₀=6.7 after 72 hours. Both ethanol and acetic acid were accumulated at 4.8 g/L. In conclusion, neoagarotetraose was produced as the target primary product by engineered yeast SB-HTU_16A_D.

### Example 8: Strain Production and Fermentation Using CRISPR-Cas9 Technology

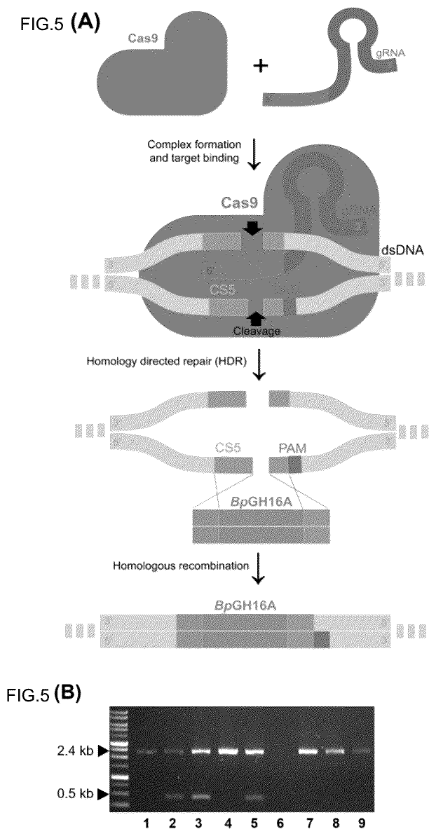

The use of genomic integration technologies, such as the CRISPR-Cas9 system, can avoid problems that may occur in complex intestinal environments when using plasmids. These problems include plasmid instability in the absence of selective pressure, potential spread to other microorganisms, and increased metabolic burden associated with the maintenance of multicopy plasmids (Durmusoglu D., et al. (2020) bioRxiv. 915389). In the present invention, BpGH16A was introduced into the genome of Saccharomyces boulardii along with SED1 for stable expression of the enzyme. BpGH16A gene insertion was performed by CRISPR-Cas9-based homologous recombination (FIG. 5A). Successful insertion of the BpGH16A gene after yeast transformation was confirmed by yeast colony PCR. A primer pair Conf-CS5-F and Conf-CS5-R was designed and used to cover 2.4-kb when the gene was inserted, and 0.5-kb when the gene was not inserted (Table 2). Successful integration of BpGH16A and SED1 into Saccharomyces boulardii was confirmed by the formation of a 2.4-kb single band in lanes 4, 7, and 8 when yeast colony PCR was performed (FIG. 5B).

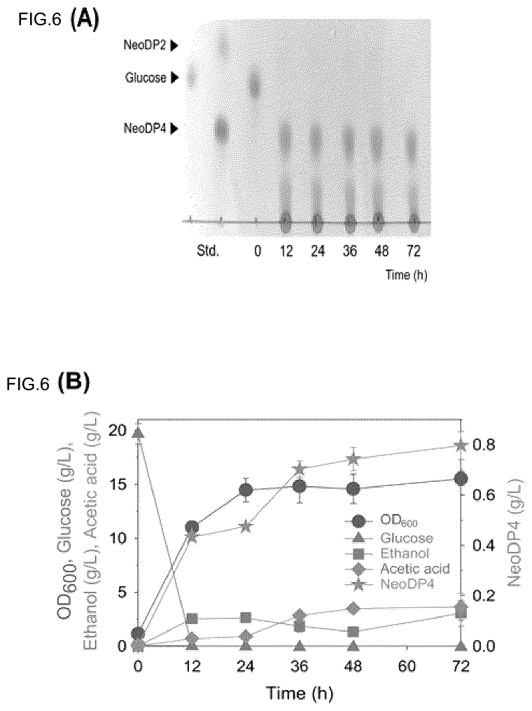

Finally, the SB_16A_D strain including BpGH16A and SED1 was constructed in the Saccharomyces boulardii genome using CRISPR-Cas9, and flask fermentation was performed for 72 hours in a YSC medium including 2.5 g/L of agarose. Neoagarotetraose production was confirmed by TLC analysis (FIG. 6A), suggesting that BpGH16A was secreted from strain SB 16A_D. For more accurate fermentation product analysis, HPLC analysis and growth measurements were also performed at each time point (FIG. 6B). Glucose was depleted before 12 hours of incubation, and the strain grew to OD₆₀₀=15.51 in 72 hours. After fermentation, 0.80 g/L of neoagarotetraose was produced, and 3.03 g/L of ethanol and 3.65 g/L of acetic acid were accumulated.

Compared to using a plasmid vector system with an auxotrophic marker, the final OD₆₀₀ after 72 hours of fermentation of the strain constructed with the CRISPR-Cas9 system was 2.3 times higher, but neoagarotetraose production was lower. The reason for this difference is estimated to be the relatively strong promoter and high copy number of the pRS426GPD plasmid (Mumberg D., et al. (1995) Gene. 156, 119-122). Nevertheless, the successful protein secretion of Saccharomyces boulardii constructed by genomic integration demonstrated the potential of Saccharomyces boulardii to be used as a microbial cell factory to produce useful proteins and substances in the human intestines.

The present invention presents a process of preparing a recombinant yeast that can produce neoagarooligosaccharides by decomposing agarose. In other words, it was found that neoagarooligosaccharides can be produced using agarose as a substrate using Saccharomyces boulardii produced through the present invention. This can be utilized to produce useful substances in the intestines, including prebiotics, using Saccharomyces boulardii, which is a probiotic yeast, and to develop recombinant enzymes.

1. Recombinant Saccharomyces boulardii transformed with a gene encoding beta-agarase.

2. The recombinant Saccharomyces boulardii of claim 1, wherein the beta-agarase gene is represented by SEQ ID NO: 1.

3. The recombinant Saccharomyces boulardii of claim 1, wherein the recombinant Saccharomyces boulardii is transformed with a gene encoding a signal peptide that secretes a beta-agarase enzyme outside strain.

4. The recombinant Saccharomyces boulardii of claim 3, wherein the signal peptide is one or more of a chicken lysozyme signal peptide (CL), a Saccharomyces cerevisiae-derived α-binding factor signal peptide (α-MF), a Saccharomyces diastaticus-derived STA1 signal peptide (STA1), and a Saccharomyces cerevisiae-derived SED1 signal peptide (SED1).

5. The recombinant Saccharomyces boulardii of claim 4, wherein the chicken lysozyme signal peptide (CL) is represented by SEQ ID NO: 2, the Saccharomyces cerevisiae-derived α-binding factor signal peptide (α-MF) is represented by SEQ ID NO: 3, the Saccharomyces diastaticus-derived STA1 signal peptide (STA1) is represented by SEQ ID NO: 4, and the Saccharomyces cerevisiae-derived SED1 signal peptide (SED1) is represented by SEQ ID NO: 5.

6. The recombinant Saccharomyces boulardii of claim 1, wherein transformation is achieved by CRISPR-Cas9 or a recombinant vector.

7. A composition for producing prebiotics comprising the recombinant Saccharomyces boulardii of claim 1 and agarose as a substrate.

8. The composition of claim 7, wherein the prebiotics are neoagarooligosaccharides.

9. A synbiotic composition comprising the recombinant Saccharomyces boulardii of claim 1 and agarose as a substrate.

10. The synbiotic composition of claim 9, further comprising probiotics.

11. A method of producing neoagarooligosaccharides, comprising:
reacting a culture fluid or extract of the recombinant Saccharomyces boulardii of claim 1 with agarose as a substrate; and
separating and purifying neoagarooligosaccharides from the product of the above step.
